(19) United States
(12) Patent Application Publication
(10) Pub. No.: US 2002/0095197 A1
(43) Pub. Date: Jul. 18, 2002

(54) Application of photochemotherapy for the treatment of cardiac arrhythmias

(76) Inventors: Albert C. Lardo, (US); Robert C. Susil, (US)

Correspondence Address: Dike, Bronstein, Roberts & Cushman, Intellectual Property Practice Group of, Edwards & Angell, LLP, P.O. Box 9169, Boston, MA, 02209, US

(21) Appl. No.: 09/904,182

(22) Filed: Jul. 11, 2001

Related U.S. Application Data
(60) Provisional application No. 60/217,522, filed on Jul. 11, 2000.

(51) Int. Cl. A61M 25/10; A61M 29/00; A61M 31/00; A61B 5/05; A61B 6/00

(52) U.S. Cl. 607/89; 604/96.01; 604/509; 600/410; 600/411; 600/433; 606/17; 604/20

(57) ABSTRACT

Methods and devices to treat and/or cure cardiac arrhythmias. The methods comprise the use of photochemotherapy or photodynamic therapy, a non-thermal method, to destroy the tissues and pathways from which abnormal signals arise and/or in other cardiac tissues such that abnormal electrical rhythms can not be generated and/or sustained.

The present application claims the benefit of U.S. provisional application No. 60/217,522, filed on Jul. 11, 2000, incorporated herein by reference in its entirety.

### FIELD OF THE INVENTION

The present invention relates to the treatment of cardiac arrhythmias and, more particularly, to methods and devices to treat and cure cardiac arrhythmias using photochemotherapy (i.e. photodynamic therapy).

### BACKGROUND OF THE INVENTION

The sinus node (SA node) is known as the heart's “natural pacemaker”. In the normal heart, electrical activation spreads in an orderly fashion from the SA, through the atria (the small, upper chambers of the heart), and into the ventricles (the large, main pumping chambers of the heart). This electrical wave acts as a signal for cardiac contraction, resulting in ejection of blood from the heart. In the normal heart, the chambers contract at a steady rhythm of about 60 to 100 beats per minute.

Without a normal pattern of electrical excitation, the heart is unable to pump efficiently. This results in irregular heartbeats called arrhythmias. In some arrhythmias, for example, the normal pattern of electrical excitation is disrupted and electrical activity proceeds through abnormal conduction pathways. In other disorders, abnormal cardiac cells may be autoarrhythmic, taking over the pacemaker action from the SA node. By ablating certain cardiac tissue, many of these arrhythmias can be eliminated.

Radiofrequency ablation is a common therapy used in the treatment of cardiac arrhythmias. In this technique, a radiofrequency catheter probe is inserted into the heart and placed on the endocardial surface of the heart in the location of the arrhythmia source. Radiofrequency energy at 550 kHz is delivered through the probe and into the tissue, which results is local resistive tissue heating and thermal damage to the tissue. This damaged tissue then undergoes irreversible cellular necrosis and can no longer conduct electrical activity, thereby terminating the arrhythmia. This approach has met with limited success in some cases of cardiac arrhythmias. In particular, radiofrequency cardiac ablation has a number of limitations when applied to atrial fibrillation, the most common and debilitating type of sustained cardiac arrhythmia known. Atrial fibrillation affects over 2 million people in the United States alone and is responsible for approximately 75,000 strokes annually. Atrial fibrillation (AF) is a rapid, irregular heart rhythm caused by abnormal electrical signals from the upper chambers of the heart (atrium). AF may increase the heart rate to and in excess of 100 to 175 beats per minute. As a result, the atria quiver rather than contracting normally, which can result in blood pooling in the atria, the formation of blood clots and strokes.

One method for catheter ablation of atrial fibrillation involves the placement of several strategically placed radiofrequency lesions that form lines of conduction block at a number of anatomically determined locations in the atria. Success has been very limited, mainly due to the inability to create, visualize and monitor linear and contiguous atrial lesions. This, in turn, leads to extremely long procedure times, ineffective treatments, and excessive x-ray exposure for both the patient and physician. Further, complications during RF ablation, such as thrombus formation and pulmonary vein stenosis, have limited the efficacy of this procedure.

In the last few years, it has been discovered that many patients with paroxysmal atrial fibrillation may have episodes of arrhythmia triggered by a focal source of rapid ectopic activity. Intracardiac mapping has demonstrated that these ectopic foci are most often located in the pulmonary veins, particularly the superior pulmonary veins, which are known to contain myocardial sleeves that may extend several centimeters from the left atrial insertions of these veins. These foci can also be a local source of sustained atrial fibrillation. A number of new techniques such as, for example, balloon ultrasound catheters, balloon laser catheters, and deployable RF catheters, have been proposed to electrically isolate the atrium and pulmonary veins in patients with focal atrial fibrillation. Focal ablation of the pulmonary veins using these energy and delivery sources, however, has experienced only limited use and success due to limitations such as (1) the inability to visualize the pulmonary vein ostia using x-ray fluoroscopy, (2) complications such as formation of systemic embolization, (3) perforation of the myocardial wall and (4) pericardial effusions and pulmonary vein stenosis caused by the aggressive post-ablation inflammatory response to high-level ostial heating and cellular neorosis.

Thus, alternative means of achieving electrical isolation from atria and pulmonary veins is needed.

Photodynamic therapy (i.e. photochemotherapy) is an emerging cancer treatment based on the combined effects of visible light and a photosensitizing agent that is activated by exposure to light of a specific wavelength. Photochemotherapy is well known (Hsi R A, Rosenthal Dl, Glatstein E. Photodynamic therapy in the treatment of cancer: current state of the art. Drugs 1999; 57(5): 725-734; Moore J V, West C M L, Whitehurst C. The biology of photodynamic therapy. Phys. Med. Biol. 1997; 42: 913-935), and, as currently performed for cancer therapy, a photosensitizing agent is injected into the patient systemically, which results in whole body tissue uptake of the agent, along with preferential uptake in the tumor. The tumor site is then illuminated with visible light of a particular energy and wavelength that is absorbed by the photosensitizing agent. This activates the photosensitizing agent, which results in the generation of cytotoxic excited state oxygen molecules in those cells in which the agent has localized. These molecules are highly reactive with cellular components and cause tumor cell death.

Photodynamic therapy initially garnered clinical interest in the mid 20^{th} century when it was demonstrated that porphyrin compounds accumulated preferentially in tumors, resulting in photosensitization and, due to the fluorescence of these compounds, aided in tumor detection. Dougherty is credited with the creation of modern photodynamic therapy, recognizing the potential of photodynamic therapy for tumor treatment and demonstrating its use in treating metastatic tumors of the skin in the 1970s (Oleinick N L, Evans H H. The photobiology of photodynamic therapy: cellular targets and mechanisms. Radiat. Res. 1998; 150: S146-56). The majority of modern research and development in photodynamic therapy has focused on the diagnosis and treatment of cancer.

### SUMMARY OF THE INVENTION

The present invention features non-thermal methods and devices for the treatment and/or cure of cardiac arrhythmias. More particularly, the present invention relates to the treatment and cure of cardiac arrhythmias using photochemotherapy or photodynamic therapy.

In accordance with methods of the present invention, photochemotherapy or photodynamic therapy can be used to destroy the tissues and pathways from which abnormal signals, leading to cardiac arrhythmias, arise. The methods of the present invention may also utilize photochemotherapy or photodynamic therapy to destroy normal tissue. For example, methods of the present invention may utilize photochemotherapy or photodynamic therapy to destroy enough normal tissue so that an arrhythmia can not be sustained. Thus, methods of the present invention may comprise the use of photochemotherapy or photodynamic therapy to destroy the tissues and pathways from which abnormal signals arise and/or to destroy other cardiac tissue such that abnormal electrical rhythms can not be sustained. In accordance with one embodiment of the invention, photochemotherapy or photodynamic therapy is used to electrically isolate the pulmonary vein from the left atrium. Preferably, this is accomplished by using photochemotherapy or photodynamic therapy to ablate at least a section of the pulmonary vein.

More particularly, a method for treating and/or curing cardiac arrhythmias comprises administering a therapeutically effective amount of a photosensitizing agent to a patient followed by exposing the patient to light capable of activating the photosensitizing agent. More specifically, the photosensitizing agent is delivered to the cardiac tissue, wherein the photosensitizing agent is preferentially absorbed by the tissues and pathways from which abnormal signals causing the arrhythmias arise and/or by normal tissues that assist in sustaining the cardiac arrhythmias. An illumination mechanism is used to activate the photosensitizing agent.

The illumination mechanism may comprise any device capable of delivering the wavelength required to activate the photosensitizing agent. In one preferred embodiment, the illumination mechanism comprises a fiberoptic catheter.

The fiberoptic catheter may designed to deliver laser fluence in a variety of illumination patterns. For example, in one embodiment, the fiberoptic catheter delivers illumination in a discrete point. In another embodiment, the fiberoptic catheter delivers illumination in a linear pattern by, for example, using a fiberoptic diffuser with the fiberoptic catheter. In yet another embodiment, the fiberoptic catheter delivers illumination in annular/ring shaped pattern by, for example, placing an angioplasty type balloon or similar mechanism over the fiberoptic.

In accordance with preferred embodiments, the photosensitizing agent is selected from porfimer sodium and phthalocyanines. The photosensitizing agent may be delivered to the cardiac tissue by a number of methods. For example, the photosensitizing agent can be delivered to the cardiac tissue systemically. Alternatively, the photosensitizing agent can be delivered to the cardiac tissue by an angioplasty catheter balloon or reservoir mechanism that is inserted to the delivery site and filled with the photosensitizing agent. The photosensitizing agent is then delivered to the cardiac tissue through, for example, pores in the balloon or reservoir or through, for example, a semipermeable membrane forming at least a portion of the balloon or reservoir. In another embodiment, the agent is perfused directly into the coronary arteries by a method similar to that used for delivering fluoroscopic contrast agent for coronary angiography.

An exemplary embodiment of the illumination device includes a catheter design wherein the photochemotherapy or photodynamic therapy is performed under either x-ray fluoroscopy or magnetic resonance (MR) imaging guidance. In a preferred embodiment, the photochemotherapy device for delivering illumination is a modified dual function internal MR imaging catheter, which combines MR imaging and photodynamic therapy. Thus, the dual function catheter can, for example, carry out photochemotherapy or photodynamic therapy, utilize MR imaging to assist in accurately positioning the catheter within the cardiac chambers and also monitor the endpoints of photodynamic therapy utilizing MR imaging.

In another embodiment, the device for photochemotherapy or photodynamic therapy of cardiac arrhythmias comprises a catheter having a balloon or reservoir at its distal end and a light source, such as a fiberoptic catheter, within the balloon or reservoir. In one embodiment, the catheter is inserted to the desired treatment site (e.g. the pulmonary vein ostia) and a photosensitizing agent is injected into the balloon or reservoir. The photosensitizing agent is then delivered by the balloon or reservoir to the treatment site, for example, through one or more pores in the balloon or reservoir, or through a semipermeable material forming at least a portion of the balloon or reservoir. The light source then delivers light through the balloon to the treatment site, thereby activating the photosensitizing agent.

The use of photochemotherapy in accordance with the present invention to treat cardiac arrhythmias offers several advantages over prior methods of treating arrhythmias.

One advantage is that photochemotherapy does not involve the destruction of tissue through either heating or freezing. Rather, photochemotherapy causes cell death through a derangement of normal cellular proteins and processes. For example, membrane transporters can be destroyed, microtubules crosslinked, or mitochondrial membrane permeability increased. Moore J V, West C M, Whitehurst C. The biology of photodynamic therapy. Phys. Med. Biol. 1997; 42 (5): 913-35. With certain photosensitizing agents and cell types, apoptotic cell death is induced. Apoptotic cell death is an orderly “programmed cell death” in which a minimal inflammatory response is produced. In comparison to thermally induced cell necrosis, apoptotic cell death helps to maintain tissue integrity, without promoting mechanical weakening or reactive tissue hyperplasia. For example, the ability of photochemotherapy to inhibit hyperplasia following vascular trauma has been demonstrated. See, e.g. Gonschoir P. Vogel-Wiens C, Goetz A E, et al. Endovascular catheter-delivered photodynamic therapy in an experimental response to injury model. Basic Res. Cardiol. 1997; 92: 310-319; Overhaus M, Heckenkamp J, Kossodo S, Leszczynski D, LaMuraglia G M. Photodynamic therapy generates a matrix barrier to invasive vascular cell migration. Circ. Res. 2000; 86: 334-340. In the effort to avoid myocardial rupture, mechanical instability, and stenosis, this is a very desirable attribute.

A further advantage of the use of photochemotherapy to treat cardiac arrhythmias is that photochemotherapy has been shown to produce crosslinking of extracellular matrix proteins, such as collagen and fibronectin. This result has been observed in the application of photochemotherapy using a phthalocyanine to prevent restenosis following angioplasty. Overhaus M, Heckenkamp J, Kossodo S, Leszczynski D, LaMuraglia G M. Photodynamic therapy generates a matrix barrier to invasive vascular cell migration. Circ. Res. 2000; 86: 334-340. In similar vascular work, Gonschoir observed the presence of inflammatory cells in the adventitia (outermost layers), but not in the media or intima (inner layers) following vascular trauma when photochemotherapy with Photofrin was applied. Gonschior P, Vogel-Wiens C, Goetz A E, et.al. Endovascular catheter-delivered photodynamic therapy in an experimental response to injury model. Basic Res. Cardiol. 1997; 92(5):310-9. The crosslinked extracellular matrix acts as a barrier to the migration of fibroblasts, myofibroblasts, and inflammatory cells, all of which are involved in tissue hypertrophy and stenosis. In cardiac ablation, it is believed that the collagen crosslinking effect of photochemotherapy will reduce post ablation hypertrophy. This is especially important in regions near the pulmonary veins, where it is particularly important to avoid stenosis.

Yet a further advantage of using photochemotherapy to treat cardiac arrhythmias is that photochemotherapy does not rely on thermal conduction effects for ablation. Cells are destroyed, via free radical generation, only within the region of illumination. This will promote sharp, well-controlled lesion borders. Moreover, lesions will be surrounded by largely undamaged myocardium, helping to preserve myocardial function. This is in contrast to RF ablation, which relies on thermal conduction and results in regions of graded tissue injury. It further is believed that photochemotherapy will enable greater success in creating continuous and uniform lesions. Using photochemotherapy, uniform illumination within the region of ablation, as opposed to uniform heating, need only be achieved. Further, modified fiber optic catheters can be used to effectively create a variety of illumination patterns suitable for various treatment sites, including continuous linear, ring shaped, and point-source areas.

Other aspects and embodiments of the invention are discussed infra.

### BRIEF DESCRIPTION OF THE DRAWINGS

FIG. 1 illustrates one embodiment of a combined internal MRI imaging and photodynamic therapy delivery catheter.

FIG. 2 illustrates the anatomy of the human heart.

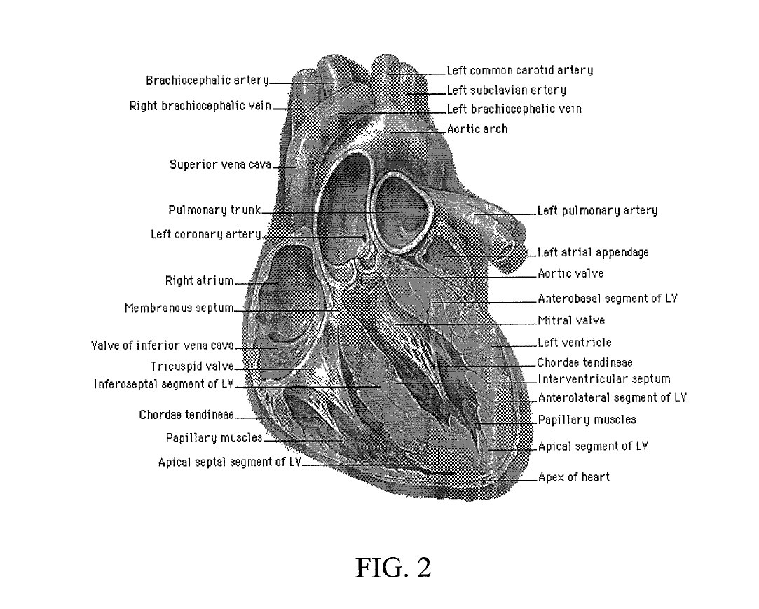

### DETAILED DESCRIPTION OF THE INVENTION

We have developed non-thermal methods and devices to treat and cure cardiac arrhythmias, including atrial fibrillation. More specifically, this method provides for the treatment and cure of cardiac arrhythmias using photochemotherapy or photodynamic therapy.

In general, in accordance with the present invention, a therapeutically effective amount of a photosensitizing agent is administered to a patient. The photosensitizing agent is preferentially absorbed by the tissues and pathways from which abnormal signals causing the arrhythmias arise and/or by normal tissues that assist in sustaining the arrhythmias. Next, the patient is exposed to light capable of activating the photosensitizing agent. Activation can be accomplished by illumination with, for example, a fiberoptic catheter. Activation of the photosensitizing agent causes cell death in those cells in which the agent has localized. For example, in one embodiment, paroxysmal atrial fibrillation is treated and/or cured by using photochemotherapy or photodynamic therapy to ablate a section of the pulmonary vein to electrically isolate the pulmonary vein from the left atrium, thereby preventing abnormal electrical signals from reaching the lower chambers of the heart.

More particularly, in accordance with the photochemotherapy or photodynamic therapy methods of the present invention, a therapeutically effective amount of a photosensitizing agent is first administered to the cardiac tissue. Preferably, the photosensitizing agent is administered approximately 4 hours before the procedure. Photosensitizing agents are well known in the art and include, by way of example, porfimer sodium (Photofrin), phthalocyanines, methoxypsoralens and porphyrins. Porfimer sodium (Photofrin) and the phthalocyanines are particularly preferred photosensitizing agents for use in the present invention.

In one embodiment, the photosensitizing agent is delivered systemically by injecting the agent into a vein. This is the most simple and widespread delivery method.

In another embodiment, the photosensitizing agent is delivered by an angioplasty catheter balloon or similar reservoir device that is inserted into the desired treatment area. Such balloons and reservoir devices are well known. Generally, an incision is first made to provide access to the desired treatment area. The balloon or reservoir is then inserted through the incision, preferably in an empty state. In a preferred embodiment, the balloon or reservoir is inserted and pressed against the myocardial wall, leading to direct application of the drug to the endocardium. Upon inserting the balloon or reservoir, the desired amount of agent is infused into the balloon or reservoir. The balloon or reservoir then delivers the agent to the treatment area. In one embodiment, one or more discrete pores are formed in the balloon or reservoir through which the agent may flow. The discrete pores may be positioned to allow for delivery of the agent to particular areas. For example, the pores may be formed in only one side of the balloon or reservoir, thereby delivering agent to only one side of the treatment area. In another embodiment, the balloon or reservoir is fabricated of a semipermeable membrane through which the agent leaches. Portions of the balloon may be fabricated of the semipermeable membrane to provide delivery of the agent to particular areas. In another embodiment, the entire balloon is fabricated of a permeable membrane and the agent to leaches from the balloon uniformly.

In a particularly preferred embodiment, the balloon or reservoir for delivering the photosensitizing agent is located at or near the distal end of a catheter, and a light source that delivers light capable of activating the photosensitizing agent is located within the balloon or reservoir. Thus, in accordance with this embodiment, the balloon laser device is inserted into the desired treatment site (e.g. the pulmonary vein ostia), photosensitizing agent is perfused into the balloon or reservoir, the balloon or reservoir delivers the photosensitizing agent to the desired treatment site, and the light source delivers light through the balloon to activate the photosensitizing agent.

In another embodiment, since the photosensitizing agent need only be delivered to the myocardium, the agent may be perfused directly into the coronary arteries by a method similar to that used for delivering fluoroscopic contrast agent for coronary angiography. This method takes advantage of the closed cardiac circulation. On first pass, the photosensitizing agent flows through the myocardium, maximizing local drug concentration.

Following administration of the photosensitizing agent, the tissue that is targeted for ablation is illuminated. Illumination can be accomplished by any device capable of providing the desired wavelength. The desired wavelengths depend on the particular photosensitizing agent used, and are well known. An incision is first made to provide access to the treatment site. In a preferred embodiment, under x-ray guidance, a standard transseptal puncture is performed to gain access to the left atrium. The catheter is then placed into the atrium and into one of the four pulmonary vein ostia. Preferably, once the illumination device engages the ostia, electrical measurements are made using, for example, electrodes placed on the device. These electrodes are preferably used to make measurements to determine whether the pulmonary veins have been electrically isolated from the left atrium. These measurements may be made at any time before, during and after the procedure to determine when electrical isolation is accomplished.

In one embodiment, the tissue is illuminated while the photosensitizing agent is being delivered, for example, while the drug is continuously transfused through the balloon or reservoir device. This could potentiate local myocardial toxicity. Alternatively, the agent can be washed out before illuminating.

In a preferred embodiment, a fiberoptic catheter is used to illuminate the tissue using non-thermal power levels of optical fluence. Using a fiberoptic catheter, laser fluence can be delivered to the endocardium in a very controlled manner, resulting in very specific, well defined patterns of cardiac ablation. Moreover, fiberoptic systems are versatile and provide great flexibility in determining the pattern of illumination.

In one embodiment, using a fiberoptic catheter, laser fluence can be delivered to a discrete point on the endocardium by abutting the end of a fiberoptic catheter with the endocardium. Thus, the end of the fiberoptic catheter may be used to illuminate precise points of tissue for precise ablation. In this embodiment, to electrically isolate the pulmonary veins from the left atrium, the fiberoptic catheter need only be rotated along the circumference of the pulmonary vein. In another embodiment, a fiberoptic diffuser can be used with the fiberoptic catheter to radiate laser fluence in a linear pattern to produce linear regions of ablated tissue. In yet another embodiment, the fiberoptic is fit with an angioplasty type balloon or similar device, which provides annular/ring shaped regions of illumination, which matches the cylindrical anatomy of the pulmonary veins.

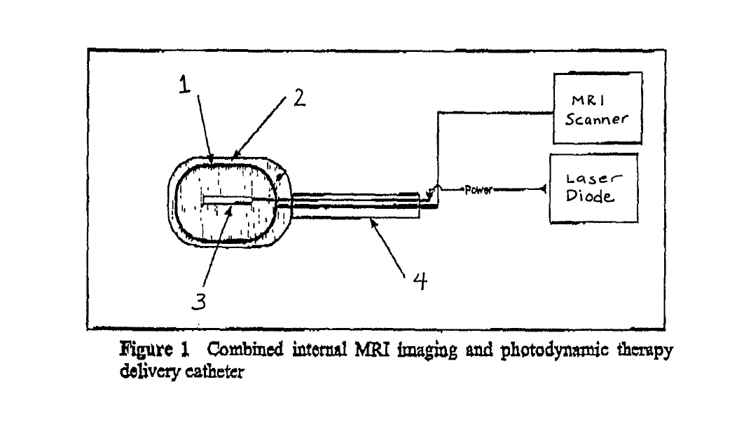

In a particularly preferred embodiment, a modified internal MR imaging catheter is used, which combines MR imaging and photodynamic therapy catheters. Such catheters are known and are described, for example, in U.S. Pat. Nos. 6,031,375, 5,928,145,and 5,928,145. Preferably, a loop or loopless internal MR imaging catheter design is employed in the present invention. These internal imaging catheters can be modified to produce a dual function catheter capable of both high-resolution imaging and photodynamic therapy. FIG., 1 is a schematic of one embodiment of an imaging and phototherapy delivery catheter in accordance with the present invention. As shown in FIG. 1, the catheter includes a single loop coil I enclosed in a catheter, such as an 8F Foley catheter, having a balloon 2 at the distal end. The balloon 2 is preferably fabricated of silicone or other flexible, biocompatible materials, and is inflated by filling it, for example, with water, saline, contrast agent, or other optically transparent solutions to allow tracking under x-ray, ultrasound, or MRI guidance. Decoupling and tuning circuitry can be attached to the coil as described, for example, in U.S. Pat. Nos. 6,031,375, 5,928,145, 5,928,145. A linear or radially diffusing laser probe fiber 3 is preferably placed coaxially with the coil 1 through the shaft 4 of the catheter and advanced to the center of the balloon 2. Energy, capable of activating the photosensitizing agent, preferably energy from about 650 nm to about 1000 nm when using porfimer sodium (Photofrin) and phthalocyanines, is scattered at the tip of the fiber 3 in a radial fashion through the balloon 2 and into the intervening tissue.

The ability to perform, high-resolution local imaging of the treatment area will be extremely useful in several ways. First, it is important to accurately position the catheter within the cardiac chambers (e.g. to position the probe in the pulmonary vein orifices). Guidance in accurately placing the catheter will preferably be based upon local anatomical landmarks and, thus, high-resolution cardiac imaging will be particularly beneficial. Further, because the procedure takes place in the left atrium, the risk of generating emboli is of particular concern. Use of local MR imaging will allow the surgeon to watch for any coagulation on the endocardial surface. Still further, MR imaging can be used to titrate and direct therapy delivery. For example, MR imaging can be used to monitor oxygenation levels, which is particularly important in photodynamic therapy because photodynamic therapy causes increased oxygen consumption. Using MR imaging, tissue oxygen saturation can be imaged (the change from diamagnetic oxyhemoglobin to paramagnetic deoxyhemoglobin results in decreased signal intensity) Foster B B, MacKay A L, Whittall K P, Kiehl K A, Smith A M, Hare R D, Liddle P F. Functional magnetic resonance imaging: the basics of blood-oxygen-level dependent (BOLD) imaging. Can Assoc Radiol J. 1998; 49:320-9. This can be used to determine which tissue is affected and also to control light intensity to ensure that tissue does not become so hypoxic as to reduce free radical generation. MR imaging can also be used to monitor phosphate levels, which is particularly important in photodynamic therapy because with photodynamic therapy, induced cellular damage, especially mitochondrial damage, rapid deterioration of ATP concentration is expected. If the mitochondrial membrane is compromised, cells have little ability to compensate for this change. Thus, MR imaging can be an excellent marker of overall cellular metabolic state and eventual response to photochemotherapy or photodynamic therapy. MR imaging can further be used to perform sodium imaging. By using photosensitizers such as Photofrin, degradation of selective membrane transport is expected. For example, in under 10 minutes, Kunz observed a strong depolarization of OK cells in vitro. Kunz L, Von Weizsacker P, Mendez F, Stark G. Radiolytic and photodynamic modifications of ion transport through the plasma membrane of OK cells: a comparison. Int J Radiat Biol. 1999;75:1029-34. Constant depolarization is associated with shifts in sodium concentration (allowing extracellular Na⁺to flow into the cytoplasmic space). Therefore, a change in sodium signal strength which is proportional to cellular depolarization/damage will be observed.

A method of treating and/or curing cardiac arrhythmias utilizing a modified internal MR imaging catheter such as that shown in FIG. 1 is as follows: approximately 4 hours before the procedure, subjects receive a photosensitizing agent administered systemically via an intravenous injection, administered via an angioplasty catheter balloon or similar reservoir device or administered by perfusing the agent directly into the coronary arteries. Under x-ray guidance, a standard transseptal puncture is performed to gain access to the left atrium. The catheter is then be placed into the atrium and into one of the four pulmonary vein ostia. Once the catheter engages the ostia, electrical measurements can be performed using electrodes placed on the outer surface of the balloon. Water, saline, contrast agent, photosensitizing agent, or other solutions are then injected into the catheter to displace the balloon and achieve circumferential ostial contact in the pulmonary vein lumen and atriovenous junction. Laser energy capable of activating the photosensitizing agent, preferably laser energy ranging from about 650 nm to about 1000 nm, more preferably, at approximately 720 nm is then delivered through the fiberoptic, preferably at a power of approximately 2 mW to approximately 1000 mW and scattered cimcumferentially into the atriovenous junction. Penetration at a laser energy of 720 nm is approximately 3-5 mm. Myocardial tissue sleeves extend several centimeters into the proximal pulmonary vein lumen and are approximately 3 mm in thickness and, thus, an energy of 720 nm is particularly suitable for use in the methods of the present invention. The laser illumination activates the photosensitizing agent, which results in cell death in those cells in which the agent has localized.

In accordance with the present method for treating aroxysmal atrial fibrillation, the pulmonary veins are electrically isolated from the left atrium utilizing photochemotherapy or photodynamic therapy methods and devices described herein, thereby preventing the abnormal electrical signals from reaching lower chambers of the heart. Preferably, to determine whether the pulmonary veins have been electrically isolated from the left atrium, electrical measurements are made. These measurements may be made at any time before, during and after the procedure to determine when electrical isolation is accomplished. These electrical measurements can be made using electrodes that are located on photochemotherapy or photodynamic therapy device.

The present invention also includes kits that comprise one or more device of the invention, preferably packaged in sterile condition. Kits of the invention also may include, for example, one or more catheter device, balloons or reservoirs, fiberoptics, photosensitizing agents, etc. for use with the device, preferably packaged in sterile condition, and/or written instructions for use of the device and other components of the kit.

All documents mentioned herein are incorporated by reference herein in their entirety.

The foregoing description of the invention is merely illustrative thereof, and it is understood that variations and modifications can be effected without departing from the scope or spirit of the invention as set forth in the following claims.

What is claimed is:

1. A non-thermal device for the treatment and/or cure of cardiac arrhythmias.

2. The non-thermal device of claim 1, wherein the non-thermal device is a photochemotherapy or photodynamic device.

3. A photochemotherapy or photodynamic therapy device for the ablation of the pulmonary vein ostia.

4. The device of claim 3, wherein the ablation is guided by MRI.

5. The device of claims 1 through 4, wherein the device includes a high resolution MRI receiver and a fiberoptic laser.

6. The device of claim 4, wherein the high resolution MRI receiver and the fiberoptic laser are housed within a balloon.

7. A device for the treatment and/or cure of cardiac arrhythmias, comprising a catheter having a balloon or reservoir at or near its distal end and a light source located within the balloon or reservoir, whereby a photosensitizing agent is perfused into and delivered by the balloon to a desired treatment site and whereby light capable of activating the photosensitizing agent is delivered by the light source through the balloon and to the desired treatment site.

8. A photochemotherapy or photodynamic therapy device for the treatment and/or cure of cardiac arrhythmias comprising:
a catheter;
a balloon at the distal end of the catheter;
a fiberoptic laser coaxial with the coil;
wherein the fiber illuminates the treatment area.

9. The device of claim 8, wherein the illumination is scattered at the tip of the fiberoptic laser radially through the balloon and into the treatment area.

10. The device of claim 8, wherein a photosensitizing agent is perfused into and delivered by the balloon to a desired treatment site.

11. The device of any one or claims 8 through 10, wherein the fiber provides illumination at a wavelength capable of activating a photosensitizing agent used in the photochemotherapy or photodynamic therapy.

12. A device for the treatment of cardiac arrhythmias comprising a dual function catheter that combines high-resolution imaging and photochemotherapy or photodynamic therapy

13. A balloon laser device for photodynamic therapy or photochemotherapy, wherein the device further provides high-resolution imaging.

14. The device of claim 13, wherein the high-resolution imaging monitors endpoints of the photodynamic therapy or photochemotherapy.

15. The device of claim 14, wherein the device further provides intravascular balloon angioplasty.

16. A device for the treatment and/or cure of cardiac arrhythmias that induces apoptotic cell death of tissues and pathways from which abnormal signals arise and/or in other cardiac tissues such that abnormal electrical rhythms can not be generated and/or sustained.

17. A device for the treatment and/or cure of cardiac arrhythmias that uses free radical generation to destroy tissues and pathways from which abnormal signals arise and/or that destroys other cardiac tissues such that abnormal electrical rhythms cannot be generated and/or sustained.

18. A medical device kit, comprising one or more of the devices of any one of claims 1 through 17.

19. The kit of claim 18, wherein the one or more devices are packaged in sterile condition.

20. A non-thermal method for treating and/or curing cardiac arrhythmias.

21. A method for treating and/or curing cardiac arrhythmias using photochemotherapy or photodynamic therapy.

22. A method to electrically isolate the pulmonary vein from the left atrium comprising using photochemotherapy or photodynamic therapy.

23. A method of ablating at least a section of the pulmonary vein using photochemotherapy or photodynamic therapy.

24. A method to treat and/or cure cardiac arrhythmias using photochemotherapy or photodynamic therapy to destroy tissues and pathways from which abnormal signals arise and/or in other cardiac tissues such that abnormal electrical rhythms can not be generated and/or sustained.

25. A photodynamic method for causing cell death in certain cardiac tissue such that abnormal electrical rhythms can not be generated and/or sustained.

26. A method to treat and/or cure cardiac arrhythmias using the device of any one of claims 1 through 17.

27. A method to treat and/or cure cardiac arrhythmias using photochemotherapy or photodynamic therapy comprising:
delivering a therapeutically effective amount of a photosensitizing agent to the cardiac tissue, wherein the photosensitizing agent is preferentially absorbed by the tissues and pathways from which abnormal signals causing the arrhythmias arise; and
activating the photosensitizing agent with an illumination mechanism.

28. The method of claim 27, wherein the step of activating the photosensitizing agent with an illumination mechanism overlaps with the step of delivering a photosensitizing agent to the cardiac tissue.

29. The method of claim 27 wherein the photosensitizing agent is selected from porfimer sodium and phthalocyanines.

30. The method of any one of claims 21 through 29, wherein the method further comprises guiding the photochemotherapy or photodynamic therapy using MRI and/or x-ray fluoroscopy.

31. The method of any one of claims 21 through 30, wherein a photosensitizing agent is delivered to the cardiac tissue systemically.

32. The method of any one of claims 21 through 30, wherein a photosensitizing agent is delivered to the cardiac tissue by an angioplasty catheter balloon or reservoir mechanism.

33. The method of claim 32, wherein the angioplasty catheter balloon or reservoir mechanism has one or more discrete pores through which the photosensitizing agent is delivered to the cardiac tissue.

34. The method of claim 33, wherein the one or more pores are positioned for delivery to a desired location in the cardiac tissue.

35. The method of claim 32, wherein at least a portion of the angioplasty catheter balloon or reservoir mechanism is fabricated of a semipermeable membrane through which the agent is delivered to the cardiac tissue.

36. The method of claim 35, wherein the portion(s) of the angioplasty catheter balloon or reservoir mechanism fabricated of the semipermeable membrane is situated to deliver the photosensitizing agent to a desired location of the cardiac tissue.

37. The method of any one of claims 21 through 30, wherein the photosensitizing agent is delivered to the cardiac tissue by directly perfusing the photosensitizing agent into the coronary arteries.

38. The method of any one of claims 19 through 37, wherein the photochemotherapy or photodynamic therapy utilizes an illumination mechanism and the illumination mechanism comprises a fiberoptic catheter.

39. The method of claim 38, wherein the fiberoptic catheter delivers illumination at a discrete point.

40. The method of claim 38, wherein the fiberoptic catheter delivers illumination in a linear pattern.

41. The method of claim 38, wherein the fiberoptic catheter delivers illumination in an annular/ring shaped pattern.

42. The method of any one of claim s 21 through 37, wherein the photochemotherapy or photodynamic therapy utilizes an illumination mechanism and the illumination mechanism comprises a dual function catheter that combines high-resolution imaging and photodynamic therapy.

43. The method of claim 42, wherein the dual function catheter comprises a balloon laser device.

44. The method of claim 43, wherein a photosensitizing agent is delivered by the balloon and the laser delivers light capable of activating the photosensitizing agent.

45. The device of any one of claims 42 through 44, further comprising the step of monitoring the endpoints of the photodynamic therapy or photochemotherapy utilizing high-resolution imaging.

46. The device of any one of claims 42 through 45, dual function catheter further provides intravascular balloon angioplasty

47. The method of any one of claims 21 through 46, further comprising the step of inserting an illumination mechanism into the treatment site and utilizing MRI to guide the illumination mechanism to the treatment site.

48. The method of any one of claims 21 through 47, further comprising the step of utilizing MR imaging to monitor coagulation on the endocardial surface.

49. The method of any one of claims 21 through 48, further comprising the step of utilizing MR imaging to monitor oxygenation levels.

50. The method of any one of claims 21 through 49, further comprising the step of utilizing MR imaging to monitor phosphate levels.

51. A method by which endpoints of photochemotherapy or photodynamic therapy are monitored by MRI and/or x-ray fluoroscopy.

52. A method for treating and/or curing cardiac arrhythmias, comprising administering a therapeutically effective amount of a photosensitizing agent to a patient followed by exposing the patient to light capable of activating the photosensitizing agent.

53. A photodynamic method for causing cell death in certain cardiac tissue such that abnormal electrical rhythms can not be generated and/or sustained.

54. A photodynamic device for causing cell death in certain cardiac tissue such that abnormal electrical rhythms can not be sustained.
